(19) United States
(12) Patent Application Publication
(10) Pub. No.: US 2014/0162359 A1
(43) Pub. Date: Jun. 12, 2014

(54) Differentiation of Human Embryonic Stem Cells into Pancreatic Endoderm

(71) Applicant: Janssen Biotech, Inc., (US)

(72) Inventor: Alireza Rezania, Raritan, NJ (US)

(73) Assignee: Janssen Biotech, Inc., Raritan, NJ (US)

(21) Appl. No.: 13/888,968

(22) Filed: May 7, 2013

Related U.S. Application Data
(60) Provisional application No. 61/643,684, filed on May 7, 2012.

(51) Int. Cl. C12N 5/071

(52) U.S. Cl. CPC C12N 5/0676
USPC 435/366; 435/377; 435/397

(57) ABSTRACT

The present invention provides methods to promote the differentiation of pluripotent stem cells. In particular, the present invention provides methods to produce a population of pancreatic endoderm cells, wherein the initial seeding density of undifferentiated epluripotent cells is defined.

### CROSS REFERENCE TO RELATED APPLICATION

The present application claims the benefit of U.S. Provisional Patent Application Ser. No. 61/643,684, filed May 7, 2012, which is incorporated herein by reference in its entirety for all purpose.

### FIELD OF THE INVENTION

The present invention is in the field of cell differentiation. More specifically, the invention provides ranges of seeding densities of human pluripotent cells and/or cells expressing markers indicative of definitive endoderm useful for subsequent efficient generation of cells expressing markers indicative of pancreatic endoderm and cells expressing markers indicative of pancreatic endocrine.

### BACKGROUND

Advances in cell-replacement therapy for Type I diabetes mellitus and a shortage of transplantable islets of Langerhans have focused interest on developing sources of insulin-producing cells, or β cells, appropriate for engraftment. One approach is the generation of functional β cells from pluripotent stem cells, such as, for example, embryonic stem cells.

In vertebrate embryonic development, a pluripotent cell gives rise to a group of cells comprising three germ layers (ectoderm, mesoderm, and endoderm) in a process known as gastrulation. Tissues such as, thyroid, thymus, pancreas, gut, and liver, will develop from the endoderm, via an intermediate stage. The intermediate stage in this process is the formation of definitive endoderm. Definitive endoderm cells express a number of markers, such as, HNF3beta, GATA4, MIXL1, CXCR4 and SOX17.

By the end of gastrulation, the endoderm is partitioned into anterior-posterior domains that can be recognized by the expression of a panel of factors that uniquely mark anterior, mid, and posterior regions of the endoderm. For example, Hhex, and Sox2 identify the anterior region while Cdx1, 2, and 4 identify the posterior half of the endoderm.

Migration of endoderm tissue brings the endoderm into close proximity with different mesodermal tissues that help in regionalization of the gut tube. This is accomplished by a plethora of secreted factors, such as FGFs, Wnts, TGF-Bs, retinoic acid (RA), and BMP ligands and their antagonists. For example, FGF4 and BMP promote Cdx2 expression in the presumptive hindgut endoderm and repress expression of the anterior genes Hhex and SOX2 (2000 Development, 127:1563-1567). WNT signaling has also been shown to work in parallel to FGF signaling to promote hindgut development and inhibit foregut fate (2007 Development, 134:2207-2217). Lastly, secreted retinoic acid by mesenchyme regulates the foregut-hindgut boundary (2002 Curr Biol, 12:1215-1220).

The level of expression of specific transcription factors may be used to designate the identity of a tissue. During transformation of the definitive endoderm into a primitive gut tube, the gut tube becomes regionalized into broad domains that can be observed at the molecular level by restricted gene expression patterns. For example, the regionalized pancreas domain in the gut tube shows a very high expression of PDX-1 and very low expression of CDX2 and SOX2. Similarly, the presence of high levels of Foxe1 are indicative of esophagus tissue; highly expressed in the lung tissue is NKX2.1; SOX2/Odd1 (OSR1) are highly expressed in stomach tissue; expression of PROX1/Hhex/AFP is high in liver tissue; SOX17 is highly expressed in biliary structure tissues; PDX1, NKX6.1/PTf1a, and NKX2.2 are highly expressed in pancreatic tissue; and expression of CDX2 is high in intestine tissue. The summary above is adapted from Dev Dyn 2009, 238:29-42 and Annu Rev Cell Dev Biol 2009, 25:221-251.

Formation of the pancreas arises from the differentiation of definitive endoderm into pancreatic endoderm (2009 Annu Rev Cell Dev Biol, 25:221-251; 2009 Dev Dyn, 238:29-42). Dorsal and ventral pancreatic domains arise from the foregut epithelium. Foregut also gives rise to the esophagus, trachea, lungs, thyroid, stomach, liver, pancreas, and bile duct system.

Cells of the pancreatic endoderm express the pancreatic-duodenal homeobox gene PDX1. In the absence of PDX1, the pancreas fails to develop beyond the formation of ventral and dorsal buds. Thus, PDX1 expression marks a critical step in pancreatic organogenesis. The mature pancreas contains, among other cell types, exocrine tissue and endocrine tissue. Exocrine and endocrine tissues arise from the differentiation of pancreatic endoderm.

D'Amouret al. describes the production of enriched cultures of human embryonic stem (ES) cell-derived definitive endoderm in the presence of a high concentration of activin and low serum (Nature Biotechnol 2005, 23:1534-1541; U.S. Pat. No. 7,704,738). Transplanting these cells under the kidney capsule of mice resulted in differentiation into more mature cells with characteristics of endodermal tissue (U.S. Pat. No. 7,704,738). Human embryonic stem cell-derived definitive endoderm cells can be further differentiated into PDX1 positive cells after addition of FGF-10 and retinoic acid (U.S. Patent Publication No. 2005/0266554A1). Subsequent transplantation of these pancreatic precursor cells in the fat pad of immune deficient mice resulted in formation of functional pancreatic endocrine cells following a 3-4 month maturation phase (U.S. Pat. No. 7,993,920 and U.S. Pat. No. 7,534,608).

Fisk et al. report a system for producing pancreatic islet cells from human embryonic stem cells (U.S. Pat. No. 7,033,831). In this case, the differentiation pathway was divided into three stages. Human embryonic stem cells were first differentiated to endoderm using a combination of sodium butyrate and activin A (U.S. Pat. No. 7,326,572). The cells were then cultured with BMP antagonists, such as Noggin, in combination with EGF or betacellulin to generate PDX1 positive cells. The terminal differentiation was induced by nicotinamide.

Small molecule inhibitors have also been used for induction of pancreatic endocrine precursor cells. For example, small molecule inhibitors of TGF-B receptor and BMP receptors (Development 2011, 138:861-871; Diabetes 2011, 60:239-247) have been used to significantly enhance number of pancreatic endocrine cells. In addition, small molecule activators have also been used to generate definitive endoderm cells or pancreatic precursor cells (Curr Opin Cell Biol 2009, 21:727-732; Nature Chem Biol 2009, 5:258-265).

Although great strides have been made in improving protocols to generate pancreatic cells from human pluripotent stem cells, there exists a great degree of variability in results reported by different groups in their efficiency of generating pancreatic cells from ES cells. This variability has been attributed to factors, such as differences in ES lines, duration of the protocol including the reagents used, and choice of adherent versus suspension cultures. We demonstrate here that whereas the efficiency in directing differentiation of ES cells into definitive endoderm is not very sensitive to the cell density, the efficiency to generate pancreatic endoderm is highly dependent on the initial seeding density of ES cells. In particular, initial cell densities in the range of 0.8−2×10⁵ cells/cm² resulted in highest expression of pancreatic endoderm and endocrine markers.

### SUMMARY

In an embodiment, the present invention concerns a method of culturing pluripotent stem cells comprising seeding the pluripotent stem cells on a surface, wherein the pluripotent stem cells are seeded at a density of from about 0.8×10⁵ cells/cm² to about 3.0×10⁵ cells/cm². In some embodiments, the pluripotent stem cells cultured are embryonic stem cells. In some embodiments, the pluripotent stem cells cultured are human embryonic stem cells. In some embodiments, the surface where the pluripotent stem cells are seeded comprises Matrigel™.

In an embodiment, the present invention relates to a method of differentiating pluripotent stem cells comprising seeding the pluripotent stem cells, at a density of from about 0.8×10⁵ cells/cm² to about 3.0×10⁵ cells/cm², on a surface, and differentiating the pluripotent stem cells into cells expressing markers indicative of definitive endoderm. In some embodiments, the pluripotent stem cells differentiated are embryonic stem cells. In some embodiments, the pluripotent stem cells differentiated are human embryonic stem cells. In some embodiments, the surface where the pluripotent stem cells are seeded comprises Matrigel™. In some embodiments, the cells expressing markers indicative of definitive endoderm are human.

In an embodiment, the invention relates to a method of obtaining cells expressing markers indicative of definitive endoderm comprising differentiating pluripotent stem cells seeded on a surface at a seeding density of from about 0.8×10⁵ cells/cm² to about 3.0×10⁵ cells/cm². In some embodiments, the pluripotent stem cells used in the method of obtaining cells expressing markers indicative of definitive endoderm are embryonic stem cells. In some embodiments, the embryonic stem cells used in the method of obtaining cells expressing markers characteristic of definitive endoderm are human embryonic stem cells. In some embodiments, the pluripotent stem cells seeded on a surface which comprises Matrigel™. In some embodiments, the cells expressing markers indicative of definitive endoderm are human.

In an embodiment, the present invention provides a method of differentiating cells expressing markers indicative of definitive endoderm comprising differentiating pluripotent stem cells that have been seeded on a first surface at a seeding density sufficient to maximize differentiation efficiency of the pluripotent stem cells into cells expressing markers indicative of definitive endoderm, and differentiating the cells expressing markers indicative of definitive endoderm into cells expressing markers indicative of pancreatic endoderm by seeding the cells expressing markers indicative of definitive endoderm on a second surface at a seeding density sufficient to maximize differentiation efficiency of the cells expressing markers indicative of definitive endoderm into cells expressing markers characteristic of pancreatic endoderm. In some aspects of the invention, the seeding density sufficient to maximize differentiation efficiency of the pluripotent stem cells into cells expressing markers indicative of definitive endoderm is from about 0.8×10⁵ cells/cm² to about 3.0×10⁵ cells/cm². In some aspects of the invention, the seeding density sufficient to maximize differentiation efficiency of the cells expressing markers indicative of definitive endoderm into cells expressing markers characteristic of pancreatic endoderm is from about 1.5×10⁵ cells/cm² to about 5.0×10⁵ cells/cm². In some embodiments, the pluripotent stem cells used in the method of differentiating cells are embryonic stem cells. In some embodiments of the invention, the embryonic stem cells used in the method of differentiating cells are human embryonic stem cells. In some embodiments of the invention, the first surface where the pluripotent stem cells are seeded comprises Matrigel™. In some embodiments of the invention, the second surface, where the cells expressing markers indicative of definitive endoderm are seeded comprises Matrigel™. In some embodiments of the invention, the cells expressing markers indicative of definitive endoderm are human. In some embodiments of the invention, the cells expressing markers indicative of pancreatic endoderm are human.

In an embodiment, the invention relates to a method of differentiating cells expressing markers indicative of definitive endoderm into cells expressing markers indicative of pancreatic endocrine comprising differentiating pluripotent stem cells that have been seeded on a surface at a seeding density of from about 0.8×10⁵ cells/cm² to about 3.0×10⁵ cells/cm² into cells expressing markers indicative of definitive endoderm, and differentiating the cells expressing markers of definitive endoderm into cells expressing markers indicative of pancreatic endocrine. In some aspects of the invention, the pluripotent stem cells used to differentiate into cells expressing markers indicative of definitive endoderm are embryonic stem cells. In some embodiments, the embryonic stem cells used to differentiate into cells expressing markers indicative of definitive endoderm are human embryonic stem cells. In some embodiments, the pluripotent stem cells used to differentiate into cells expressing markers indicative of definitive endoderm are seeded on a surface which comprises Matrigel™.

In an embodiment, the invention relates to a method of obtaining cells expressing markers indicative of pancreatic endoderm comprising seeding pluripotent stem cells on a surface, differentiating the pluripotent stem cells into cells expressing markers indicative of definitive endoderm, seeding the cells expressing markers indicative of definitive endoderm on a surface, and differentiating the cells expressing markers indicative of definitive endoderm into cells expressing markers indicative of pancreatic endoderm. In some aspects of the invention, the pluripotent stem cells used in the method of obtaining cells expressing markers indicative of pancreatic endoderm are seeded on the surface at a density of from about 0.8×10⁵ cells/cm² to about 3.0×10⁵ cells/cm². In some aspects of the invention, the cells expressing markers indicative of definitive endoderm are seeded on a surface at a density of from about 1.5×10⁵ cells/cm² to about 5.0×10⁵ cells/cm². In some aspects of the invention, the pluripotent stem cells differentiated into cells expressing markers indicative of definitive endoderm are embryonic stem cells. In some aspects of the invention, the embryonic stem cells differentiated into cells expressing markers indicative of definitive endoderm are human embryonic stem cells. In some aspects of the invention the pluripotent stem cells are seeded on a surface comprising Matrigel™. In some aspects of the invention, the cells expressing markers indicative of definitive endoderm are seeded on a surface comprising Matrigel™.

In one embodiment, the invention relates to a method of obtaining cells expressing markers indicative of pancreatic endocrine comprising seeding pluripotent stem cells on a surface; differentiating the pluripotent stem cells into cells expressing markers indicative of the definitive endoderm; and differentiating the cells expressing markers indicative of definitive endoderm into cells expressing markers indicative of pancreatic endocrine. In some aspects of the invention, the pluripotent stem cells used in the method of obtaining cells expressing markers indicative of pancreatic endoderm are seeded on the surface at a density of from about 0.8×10⁵ cells/cm² to about 3.0×10⁵ cells/cm². In some aspects of the invention, the cells expressing markers indicative of definitive endoderm are seeded on a surface at a density of from about 1.5×10⁵ cells/cm² to about 5.0×10⁵ cells/cm². In some aspects of the invention, the pluripotent stem cells differentiated into cells expressing markers indicative of definitive endoderm are embryonic stem cells. In some aspects of the invention, the embryonic stem cells differentiated into cells expressing markers indicative of definitive endoderm are human embryonic stem cells. In some aspects of the invention the pluripotent stem cells are seeded on a surface comprising Matrigel™. In some aspects of the invention, the cells expressing markers indicative of definitive endoderm are seeded on a surface comprising Matrigel™.

In an embodiment, the invention relates to a method of differentiating cells expressing markers indicative of definitive endoderm comprising seeding cells expressing markers indicative of definitive endoderm on a surface at a seeding density of from about 1.5×10⁵ cells/cm² to about 5.0×10⁵ cells/cm², and differentiating the cells expressing markers indicative of definitive endoderm into cells expressing markers indicative of pancreatic endoderm. In some aspects of the invention, the cells used are human.

The present invention provides a population of cells expressing markers indicative of the pancreatic endoderm lineage obtained in vitro by the stepwise differentiation of 0.8×10⁵ pluripotent cells/cm² to 3×10⁵ pluripotent cells/cm².

In an embodiment of the present invention, cells expressing markers indicative of pancreatic endocrine lineage are obtained in vitro by the stepwise differentiation of 0.8×10⁵ pluripotent cells/cm² to 3×10⁵ pluripotent cells/cm².

In an embodiment of the present invention, cells expressing markers indicative of pancreatic endoderm lineage are obtained in vitro by the stepwise differentiation of cells expressing markers indicative of the definitive endoderm seeded on a surface at a density of 1.5×10⁵ cells/cm² to 5×10⁵ cells/cm².

In an embodiment of the present invention, cells expressing markers indicative of pancreatic endocrine lineage are obtained in vitro by the stepwise differentiation of cells expressing markers indicative of definitive endoderm seeded on a surface at 1.5×10⁵ to 5×10⁵ cells/cm².

### BRIEF DESCRIPTION OF THE DRAWINGS

FIG. 1A to FIG. 1F shows FACS histogram expression profiles of CXCR4 (Y-axis, marker of DE) and CD-9 (X-axis, marker of undifferentiated ES cells) for H1 cells seeded at 0.3×10⁵ cells/cm² (FIG. 1A), 0.75×10⁵ cells/cm² (FIG. 1B), 1×10⁵ cells/cm² (FIG. 1C), 1.5×10⁵ cells/cm² (FIG. 1D), 1.8×10⁵ cells/cm² (FIG. 1E), and 2×10⁵ cells/cm² (FIG. 1F).

FIG. 2A to FIG. 2G show data from real-time PCR analyses of the expression of the following genes in cells of the human embryonic stem cell line H1 seeded at various densities and subsequently differentiated to DE as outlined in Example 1: SOX7 (FIG. 2A), NANOG (FIG. 2B), OCT4 (FIG. 2C), AFP (FIG. 2D), SOX17 (FIG. 2E), FOXA2 (FIG. 2F), and CXCR4 (FIG. 2G).

FIG. 3A-3H show phase contrast images of cultures prior to induction of DE that were seeded at various cell densities: 0.3×10⁵ cells/cm² (FIG. 3A), 0.5×10⁵ cells/cm² (FIG. 3B), 0.75×10⁵ cells/cm² (FIG. 3C), 0.9×10⁵ cells/cm² (FIG. 3D), 1×10⁵ cells/cm² (FIG. 3E), 1.1×10⁵ cells/cm² (FIG. 3F), 1.2×10⁵ cells/cm² (FIG. 3G) and 1.5×10⁵ cells/cm² (FIG. 3H).

FIG. 4A-4G show phase contrast images of DE day 4 cultures that were initially seeded at various cell densities of ES cells: 0.3×10⁵ cells/cm² (FIG. 4A), 0.5×10⁵ cells/cm² (FIG. 4B), 0.75×10⁵ cells/cm² (FIG. 4C), 1×10⁵ cells/cm² (FIG. 4D), 1.1×10⁵ cells/cm² (FIG. 4E), 1.2×10⁵ cells/cm² (FIG. 4F) and 1.5×10⁵ cells/cm² (FIG. 4G).

FIG. 5A-5F show phase contrast images of stage 5 cultures that were initially seeded at various cell densities of ES cells: 5×10⁴ cells/cm² (FIG. 5A), 7.5×10⁴ cells/cm² (FIG. 5B), 1×10⁵ cells/cm² (FIG. 5C), 1.5×10⁵ cells/cm² (FIG. 5D), 1.8×10⁵ cells/cm² (FIG. 5E) and 2.0×10⁵ cells/cm² (FIG. 5F).

FIG. 6A to FIG. 6J depict data from real-time PCR analyses of the expression of the following genes in cells of the human embryonic stem cell line H1 seeded at various densities and subsequently differentiated to stage 5 as outlined in Example 2: ZIC1 (FIG. 6A), CDX2 (FIG. 6B), PDX-1 (FIG. 6C), NKX6.1 (FIG. 6D), NKX2.2 (FIG. 6E), NGN3 (FIG. 6F), NEUROD (FIG. 6G), insulin (FIG. 6H) HNF4a (FIG. 6I), and PTF1a (FIG. 6J).

### DETAILED DESCRIPTION

For clarity of disclosure, and not by way of limitation, the detailed description of the invention is divided into the following subsections that describe or illustrate certain features, embodiments or applications of the present invention.

### DEFINITIONS

Stem cells are undifferentiated cells defined by their ability, at the single cell level, to both self-renew and differentiate. Stem cells may produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm). Stem cells also give rise to tissues of multiple germ layers following transplantation and contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages but all within a particular tissue, organ, or physiological system (for example, hematopoietic stem cells (HSC) can produce progeny that include HSC (self-renewal), blood cell restricted oligopotent progenitors, and all cell types and elements (e.g., platelets) that are normal components of the blood); (4) oligopotent, meaning able to give rise to a more restricted subset of cell lineages than multipotent stem cells; and (5) unipotent, meaning able to give rise to a single cell lineage (e.g., spermatogenic stem cells).

Differentiation is the process by which an unspecialized (“uncommitted”) or less specialized cell acquires the features of a specialized cell such as, for example, a nerve cell or a muscle cell. A differentiated cell or a differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

“Markers”, as used herein, are nucleic acid or polypeptide molecules that are differentially expressed in a cell of interest. In this context, differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive for” a specific marker or “positive” when the specific marker is detected in the cell. Similarly, the cell is “negative for” a specific marker, or “negative” when the specific marker is not detected in the cell.

As used herein, “Cell density” and “Seeding Density” are used interchangeably herein and refer to the number of cells seeded per unit area of a planar or curved substrate.

As used herein, “stage 1” and “S1” are used interchangeably to identify cells expressing markers characteristic of the definitive endoderm (DE).

“Definitive endoderm”, as used herein, refers to cells which bear the characteristics of cells arising from the epiblast during gastrulation and which form the gastrointestinal tract and its derivatives. Definitive endoderm cells express at least one of the following markers: HNF3 beta, GATA4, SOX17, CXCR4, Cerberus, OTX2, goosecoid, C-Kit, CD99, and MIXL1.

“Gut tube”, as used herein, refers to cells derived from definitive endoderm that express at least one of the following markers: HNF3-beta, HNF1-beta, or HNF4-alpha. Gut tube cells can give rise to all endodermal organs, such as lungs, liver, pancreas, stomach, and intestine.

Used herein interchangeably are “stage 2” and “S2” which identify cells expressing markers characteristic of the primitive gut tube.

“Foregut endoderm” refers to endoderm cells that give rise to esophagus, lungs, stomach, liver, pancreas, gall bladder, and a portion of the duodenum.

“Posterior foregut” refers to endoderm cells that can give rise to posterior stomach, pancreas, liver, and a portion of the duodenum.

“Mid-gut endoderm” refers to endoderm cells that can give rise to the intestines, portions of the duodenum, appendix, and ascending colon.

“Hind-gut endoderm” refers to endoderm cells that can give rise to the distal third of the transverse colon, the descending colon, sigmoid colon and rectum.

Both “stage 3” and “S3” are used interchangeably to identify cells expressing markers characteristic of the foregut endoderm. “Cells expressing markers characteristic of the foregut lineage”, as used herein, refers to cells expressing at least one of the following markers: PDX-1, FOXA2, CDX2, SOX2, and HNF4 alpha.

Used interchangeably herein are “stage 4” and “S4” to identify cells expressing markers characteristic of the pancreatic foregut precursor. “Cells expressing markers characteristic of the pancreatic foregut precursor lineage”, as used herein, refers to cells expressing at least one of the following markers: PDX-1, NKX6.1, HNF6, FOXA2, PTF1a, Prox1 and HNF4 alpha.

As used herein, “stage 5” and “S5” are used interchangeably to identify cells expressing markers characteristic of the pancreatic endoderm and pancreatic endocrine precursor cells. “Cells expressing markers characteristic of the pancreatic endoderm lineage”, as used herein, refers to cells expressing at least one of the following markers: PDX1, NKX6.1, HNF1 beta, PTF1 alpha, HNF6, HNF4 alpha, SOX9, HB9 or PROX1. Cells expressing markers characteristic of the pancreatic endoderm lineage do not substantially express CDX2 or SOX2.

“Pancreatic endocrine cell”, or “Pancreatic hormone expressing cell”, or “Cells expressing markers characteristic of the pancreatic endocrine lineage” as used herein, refers to a cell capable of expressing at least one of the following hormones: insulin, glucagon, somatostatin, ghrelin, and pancreatic polypeptide.

“Pancreatic endocrine precursor cell” or “Pancreatic endocrine progenitor cell” refers to pancreatic endoderm cells capable of becoming a pancreatic hormone expressing cell. Such a cell can express at least one of the following markers: NGN3, NKX2.2, NeuroD, ISL-1, Pax4, Pax6, or ARX.

Used interchangeably herein are “d1”, “d1”, and “day 1”; “d2”, “d 2”, and “day 2”; “d3”, “d 3”, and “day 3”, and so on. These number letter combinations specify the day of incubation in the different stages during the stepwise differentiation protocol of the instant application.

“Glucose” and “D-Glucose” are used interchangeably herein and refer to dextrose, a sugar commonly found in nature.

Used interchangeably herein are “NeuroD” and “NeuroD1” which identify a protein expressed in pancreatic endocrine progenitor cells and the gene encoding it.

Used interchangeably herein are “LDN” and “LDN-193189” to indicate a BMP receptor inhibitor available from Stemgent, CA, USA.

### Isolation, Expansion and Culture of Pluripotent Stem Cells

Pluripotent stem cells may express one or more of the stage-specific embryonic antigens (SSEA) 3 and 4, and markers detectable using antibodies designated Tra-1-60 and Tra-1-81 (Thomson et al. 1998, Science 282:1145-1147). Differentiation of pluripotent stem cells in vitro results in the loss of SSEA-4, Tra-1-60, and Tra-1-81 expression. Undifferentiated pluripotent stem cells typically have alkaline phosphatase activity, which can be detected by fixing the cells with 4% paraformaldehyde, and then developing with Vector Red as a substrate, as described by the manufacturer (Vector Laboratories, CA, USA). Undifferentiated pluripotent stem cells also typically express OCT4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of stem cells can be confirmed, for example, by injecting cells into SCID mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered. Pluripotent cells may be readily expanded in culture using various feeder layers or by using matrix protein coated vessels. Alternatively, chemically defined surfaces in combination with defined media such as mTesr®1 media (StemCell Technologies, Vancouver, Canada) may be used for routine expansion of the cells. Pluripotent cells may be readily removed from culture plates using enzymatic, mechanical or use of various calcium chelators such as EDTA (Ethylenediaminetetraacetic acid). Alternatively, pluripotent cells may be expanded in suspension in the absence of any matrix proteins or a feeder layer.

### Sources of Pluripotent Stem Cells

The types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily, before approximately 10 to 12 weeks gestation. Non-limiting examples are established lines of human embryonic stem cells (hESCs) or human embryonic germ cells, such as, for example the human embryonic stem cell lines H1, H7, and H9 (WiCell Research Institute, Madison, Wis., USA). Also suitable are cells taken from a pluripotent stem cell population already cultured in the absence of feeder cells. Also suitable are inducible pluripotent cells (IPS) or reprogrammed pluripotent cells that can be derived from adult somatic cells using forced expression of a number of pluripotent related transcription factors, such as OCT4, NANOG, Sox2, KLF4, and ZFP42 (Annu Rev Genomics Hum Genet 2011, 12:165-185). The human embryonic stem cells used in the methods of the invention may also be prepared as described by Thomson et al. (U.S. Pat. No. 5,843,780; Science, 1998, 282:1145-1147; Curr Top Dev Biol 1998, 38:133-165; Proc Natl Acad Sci U.S.A. 1995, 92:7844-7848).

### Formation of Cells Expressing Markers Characteristic of the Pancreatic Endoderm Lineage from Pluripotent Stem Cells

Characteristics of pluripotent stem cells are well known to those skilled in the art, and additional characteristics of pluripotent stem cells continue to be identified. Pluripotent stem cell markers include, for example, the expression of one or more of the following: ABCG2, cripto, FOXD3, CONNEXIN43, CONNEXIN45, OCT4, SOX2, NANOG, hTERT, UTF1, ZFP42, SSEA-3, SSEA-4, Tra 1-60, Tra 1-81.

Pluripotent stem cells suitable for use in the present invention include, for example, the human embryonic stem cell line H9 (NIH code: WA09), the human embryonic stem cell line H1 (NIH code: WA01), the human embryonic stem cell line H7 (NIH code: WA07), and the human embryonic stem cell line SA002 (Cellartis, Sweden). Also suitable for use in the present invention are cells that express at least one of the following markers characteristic of pluripotent cells: ABCG2, cripto, CD9, FOXD3, CONNEXIN43, CONNEXIN45, OCT4, SOX2, NANOG, hTERT, UTF1, ZFP42, SSEA-3, SSEA-4, Tra 1-60, and Tra 1-81.

Markers characteristic of the definitive endoderm lineage are selected from the group consisting of SOX17, GATA4, HNF3 beta, GSC, CERT, Nodal, FGF8, Brachyury, Mix-like homeobox protein, FGF4, CD48, eomesodermin (EOMES), DKK4, FGF17, GATA6, CXCR4, C-Kit, CD99, and OTX2. Suitable for use in the present invention is a cell that expresses at least one of the markers characteristic of the definitive endoderm lineage. In one aspect of the present invention, a cell expressing markers characteristic of the definitive endoderm lineage is a primitive streak precursor cell. In an alternate aspect, a cell expressing markers characteristic of the definitive endoderm lineage is a mesendoderm cell. In an alternate aspect, a cell expressing markers characteristic of the definitive endoderm lineage is a definitive endoderm cell.

Markers characteristic of the pancreatic endoderm lineage are selected from the group consisting of PDX1, NKX6.1, HNF1 beta, PTF1 alpha, HNF6, HNF4 alpha, SOX9, HB9 and PROX1. Suitable for use in the present invention is a cell that expresses at least one of the markers characteristic of the pancreatic endoderm lineage. In one aspect of the present invention, a cell expressing markers characteristic of the pancreatic endoderm lineage is a pancreatic endoderm cell wherein the expression of PDX-1 and NKX6.1 are substantially higher than the expression of CDX2 and SOX2.

Markers characteristic of the pancreatic endocrine lineage are selected from the group consisting of NGN3, NEUROD, ISL1, PDX1, NKX6.1, PAX4, ARX, NKX2.2, and PAX6. In one embodiment, a pancreatic endocrine cell is capable of expressing at least one of the following hormones: insulin, glucagon, somatostatin, and pancreatic polypeptide. Suitable for use in the present invention is a cell that expresses at least one of the markers characteristic of the pancreatic endocrine lineage. In one aspect of the present invention, a cell expressing markers characteristic of the pancreatic endocrine lineage is a pancreatic endocrine cell. The pancreatic endocrine cell may be a pancreatic hormone-expressing cell. Alternatively, the pancreatic endocrine cell may be a pancreatic hormone-secreting cell.

In one aspect of the present invention, the pancreatic endocrine cell is a cell expressing markers characteristic of the β cell lineage. A cell expressing markers characteristic of the β cell lineage expresses PDX1 and at least one of the following transcription factors: NKX2.2, NKX6.1, NEUROD, ISL1, HNF3 beta, MAFA, PAX4, and PAX6. In one aspect of the present invention, a cell expressing markers characteristic of the β cell lineage is a β cell.

The present invention recites a method of culturing human pluripotent stem cells comprising seeding human pluripotent stem cells on a surface at a density of from about 0.8×10⁵ cells/cm² to about 3.0×10⁵ cells/cm². In one aspect of the invention, the human pluripotent stem cells are human embryonic stem cells. In some aspects of the invention the surface where the cells are seeded comprises Matrigel™.

In one aspect, the invention refers to a method of differentiating pluripotent stem cells. The method comprises seeding the pluripotent stem cells at a density of from about 0.8×10⁵ cells/cm² to about 3.0×10⁵ cells/cm² on a surface and then differentiating the pluripotent cells into cells expressing markers indicative of definitive endoderm. In some aspects of the invention, the pluripotent cells are embryonic stem cells. In some aspects of the invention, the embryonic stem cells are human embryonic stem cells. In some aspects of the invention the surface where the cells are seeded comprises Matrigel™.

The invention refers to a method of obtaining cells expressing markers indicative of definitive endoderm by differentiating human embryonic pluripotent stem cells that have been seeded on a surface at a seeding density of from about 0.8×10⁵ cells/cm² to about 3.0×10⁵ cells/cm². In some aspects of the invention the surface where the cells are seeded comprises Matrigel™.

In one aspect, the invention refers to a method of differentiating cells expressing markers indicative of the human definitive endoderm comprising differentiating human embryonic pluripotent stem cells, that have been seeded on a first surface at a seeding density sufficient to maximize differentiation of the pluripotent cells, into cells expressing markers indicative of the definitive endoderm; and differentiating the cells expressing markers indicative of definitive endoderm, seeded on a second surface at a seeding density sufficient to maximize the differentiation efficiency, into cells expressing markers indicative of pancreatic endoderm. In some embodiments, the pluripotent stem cells are seeded at a seeding density of from about 0.8×10⁵ cells/cm² to about 3.0×10⁵ cells/cm². In some embodiments, the cells expressing markers indicative of definitive endoderm are seeded on the surface at a seeding density of from about from about 1.5×10⁵ cells/cm² to about 5.0×10⁵ cells/cm². In some aspects, the pluripotent cells in the method of differentiating cells expressing markers indicative of the human definitive endoderm comprises using embryonic stem cells. In some aspects of the invention, the embryonic stem cells are human embryonic stem cells. In some aspects of the invention the surfaces where the cells are seeded comprise Matrigel™.

The invention refers to a method of differentiating cells expressing markers indicative of definitive endoderm that have been produced by the differentiation of pluripotent stem cells into cells expressing markers indicative of pancreatic endocrine. Where the pluripotent stem cells have been seeded on a surface at a seeding density of from about 0.8×10⁵ cells/cm² to about 3.0×10⁵ cells/cm². In some aspects of the invention the pluripotent stem cells used are embryonic stem cells. In some aspects of the invention, the embryonic stem cells used are human embryonic stem cells. In some aspects of the invention the surfaces where the cells are seeded comprise Matrigel™.

In one aspect, the invention refers to a method of obtaining cells expressing markers indicative of pancreatic endoderm comprising seeding pluripotent stem cells on a surface; differentiating the pluripotent stem cells into cells expressing markers indicative of the definitive endoderm; and differentiating the cells expressing markers indicative of the definitive endoderm into cells expressing markers indicative of pancreatic endoderm. In some aspects of the invention, the pluripotent stem cells are seeded at density of from about 0.8×10⁵ cells/cm² to about 3.0×10⁵ cells/cm². In some aspects of the invention, the cells expressing markers indicative of definitive endoderm are seeded at a density of from about 1.5×10⁵ cells/cm² to about 5.0×10⁵ cells/cm². In some aspects of the invention, the pluripotent stem cells are embryonic stem cells. In some aspects of the invention, the embryonic stem cells are human embryonic stem cells. In some aspects of the invention the surfaces where the cells are seeded comprise Matrigel™.

In one aspect, the invention relates to a method of obtaining cells expressing markers indicative of pancreatic endocrine lineage, comprising seeding pluripotent stem cells on a surface; differentiating the pluripotent stem cells into cells expressing markers indicative of definitive endoderm; and differentiating the cells expressing markers indicative of definitive endoderm into cells expressing markers indicative of pancreatic endocrine. In some aspects of the invention, the pluripotent stem cells used to obtain cells expressing markers indicative of pancreatic endocrine lineage are seeded at a density of from about 0.8×10⁵ cells/cm² to about 3.0×10⁵ cells/cm². In some aspects of the invention, the cells expressing markers indicative of definitive endoderm are seeded at a density of from about 1.5×10⁵ cells/cm² to about 5.0×10⁵ cells/cm². In some aspects of the invention, the pluripotent stem cells are embryonic stem cells. In some aspects of the invention, the embryonic stem cells are human embryonic stem cells. In some aspects of the invention the surfaces where the cells are seeded comprise Matrigel™.

In one aspect, the invention refers to a method of differentiating cells expressing markers indicative of definitive endoderm comprising seeding cells expressing markers indicative of definitive endoderm on a surface at a seeding density of from about 1.5×10⁵ cells/cm² to about 5.0×10⁵ cells/cm² and then differentiating the cells expressing markers indicative of definitive endoderm into cells expressing markers indicative of pancreatic endoderm. In some aspects of the invention, the cells expressing markers indicative of definitive endoderm used in the method are human cells expressing markers indicative of definitive endoderm. In some aspects of the invention, the cells expressing markers indicative of pancreatic endoderm are human.

In one aspect, the invention relates to a method of differentiating cells expressing markers indicative of definitive endoderm seeded on a surface at a seeding density of from about 1.5×10⁵ cells/cm² to about 5.0×10⁵ cells/cm² and then differentiating the cells expressing markers indicative of definitive endoderm into cells expressing markers indicative of pancreatic endocrine. In some aspects, the cells expressing markers indicative of the definitive endoderm are human. In some aspects, the cells expressing markers indicative of the pancreatic endocrine are human.

This invention describes a range of ES cell densities that can be efficiently differentiated to pancreatic endoderm and endocrine lineages.

Another aspect of this invention describes a range of DE cell densities that can be efficiently differentiated to pancreatic endoderm and endocrine lineages.

Publications cited throughout this document are hereby incorporated by reference in their entirety. The present invention is further illustrated, but not limited, by the following examples.

### EXAMPLES

### Example 1

### Seeding Density of Embryonic Stem Cells does not Significantly Affect Expression of Definitive Endoderm Markers

This example was carried out to understand if the initial seeding density of ES cells would significantly impact production of cells of the definitive endoderm lineage.

Cells of the human embryonic stem cell line H1 (hESC H1) were harvested at various passages (passage 40 to passage 52) and were seeded as single cells at the following densities: 0.3×10⁵ cells/cm², 0.5×10⁵ cells/cm², 0.75×10⁵ cells/cm², 0.9×10⁵ cells/cm², 1×10⁵ cells/cm², 1.25×10⁵ cells/cm², 1.5×10⁵ cells/cm², 1.8×10⁵ cells/cm², and 2×10⁵ cells/cm² on Matrigel™ (1:30 dilution; BD Biosciences, Franklin Lakes, N.J.) coated dishes in either mTeSR®1 media (StemCell Technologies, Vancouver, Canada) or MEF-CM (conditioned media) supplemented with 10 μM of Y27632 (Rock inhibitor, Catalog No. Y0503, SigmaAldrich, St. Louis, Mo.). Forty-eight hours post seeding, cultures were washed and incubated in incomplete PBS (phosphate buffered saline without Mg or Ca) for approximately 30 seconds. Cultures were differentiated into definitive endoderm (DE) lineage as follows:

Stage 1 (Definitive Endoderm (DE)-4 days): Cells were cultured for one day in stage 1 media: MCDB-131 medium (Catalog No. 10372-019, Invitrogen, Carlsbad, Calif.) supplemented with 2% fatty acid-free BSA (Catalog No. 68700, Proliant, Ankeny, Iowa), 0.0012 g/ml sodium bicarbonate (Catalog No. 53187, SigmaAldrich), 1× GlutaMax™ (Catalog No. 35050-079, Invitrogen), 2.5 mM D-Glucose (Catalog No. G8769, SigmaAldrich), 1:50000×ITS-X (Invitrogen), 100 ng/ml GDF8 (R&D Systems, Minneapolis, Minn.) and 2.5 μM MCX compound (a GSK3B inhibitor, 14-Prop-2-en-1-yl-3,5,7,14,17,23,27-heptaazatetracyclo [19.3.1.1˜2,6˜0.1˜8,12˜]heptacosa-1(25),2(27),3,5,8(26),9,11,21,23-nonaen-16-one, US Patent Application Publication No. 2010-0015711; incorporated herein by reference in its entirety). Cells were then cultured for additional three days in MCDB-131 medium supplemented with 2% fatty acid-free BSA, 0.0012 g/ml sodium bicarbonate, 1× GlutaMax™, 2.5 mM D-Glucose, 100 ng/ml GDF8, and 1:50000×ITS-X.

At end of DE stage, samples were collected and analyzed by real-time PCR and fluorescent activated cell sorting (FACS). Cell hESC-derived cells were released into single-cell suspension by incubation in TrypLE Express (Invitrogen Catalog No. 12604) at 37° C. for 3-5 minutes and subsequently counted in duplicates using a hemocytometer. Cells were then washed twice in staining buffer (PBS containing 0.2% BSA) (BD Biosciences Catalog No. 554657). For surface marker staining, 1×10⁵ to 1×10⁶ cells were re-suspended in 100 μl blocking buffer (0.5% human gamma-globulin diluted 1:4 in staining buffer). Directly conjugated primary antibodies CD184 APC (Allophycocyanin, BD Biosciences Catalog No. 555976), and CD9 PE (BD Biosciences Catalog No. 555372) were added to the cells at a final dilution of 1:20 and incubated for 30 minutes at 4° C. Stained cells were washed twice in BD staining buffer, re-suspended in 200 μl staining buffer, followed by incubation in 15 μl of 7AAD for live/dead discrimination prior to analysis on the BD FACS Canto.

Total RNA was extracted with the RNeasy Mini Kit (Qiagen; Valencia, Calif.) and reverse-transcribed using a High Capacity cDNA Reverse Transcription Kit (Applied Biosystems, Foster City, Calif.) according to manufacturer's instructions. cDNA was amplified using Taqman Universal Master Mix and Taqman Gene Expression Assays which were pre-loaded onto custom Taqman Arrays (Applied Biosystems). Data were analyzed using Sequence Detection Software (Applied Biosystems) and normalized to undifferentiated human embryonic stem (hES) cells using the ΔΔCt method. All primers were purchased from Applied Biosystems.

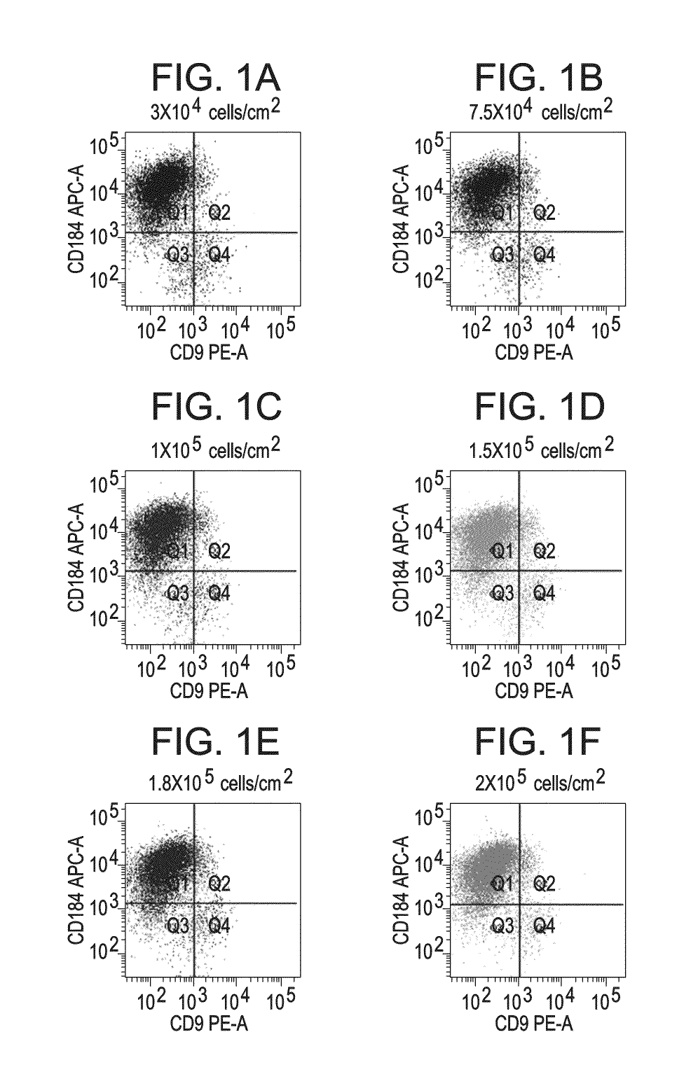

FIG. 1A to FIG. 1F shows FACS histogram expression profiles of CXCR4 (Y-axis, marker of DE) and CD-9 (X-axis, marker of undifferentiated ES cells) for H1 cells seeded at 0.3×10⁵ cells/cm² (FIG. 1A), 0.75×10⁵ cells/cm² (FIG. 1B), 1×10⁵ cells/cm² (FIG. 1C), 1.5×10⁵ cells/cm² (FIG. 1D), 1.8×10⁵ cells/cm² (FIG. 1E), and 2×10⁵ cells/cm² (FIG. 1F). Percentage expression of CXCR4 and CD9 is summarized in Table I. As shown in FIG. 1 and Table I, the initial seeding density of undifferentiated ES cells had no significant impact on subsequent differentiation to definitive endoderm as measured by upregulation of CXCR4 and down regulation of CD9.

TABLE I
Effect of Seeding Density of ES Cells on Expression of Definitive
Endoderm Marker CXCR4

| Seeding density of | DE day 0 | DE day 4 | | |
|---|---|---|---|---|
| ES cells | Cell density | Cell density | | |
| (cells/cm²) | (cells/cm²) | (cells/cm²) | % CXCR4 | % CD9 |
| 0.5 × 10⁴ | 1.1 | 2.6 | 93.3 | 4.9 |
| 0.75 × 10⁴ | 1.25 | 2.8 | 93.1 | 5.6 |
| 1.0 × 10⁵ | 2.23 | 3.95 | 93.1 | 5.3 |
| 1.5 × 10⁵ | 2.87 | 3.75 | 90.9 | 6.5 |
| 1.8 × 10⁵ | 2.58 | 4.4 | 93.1 | 4.7 |
| 2.0 × 10⁵ | 2.8 | 5.2 | 92.2 | 6.1 |

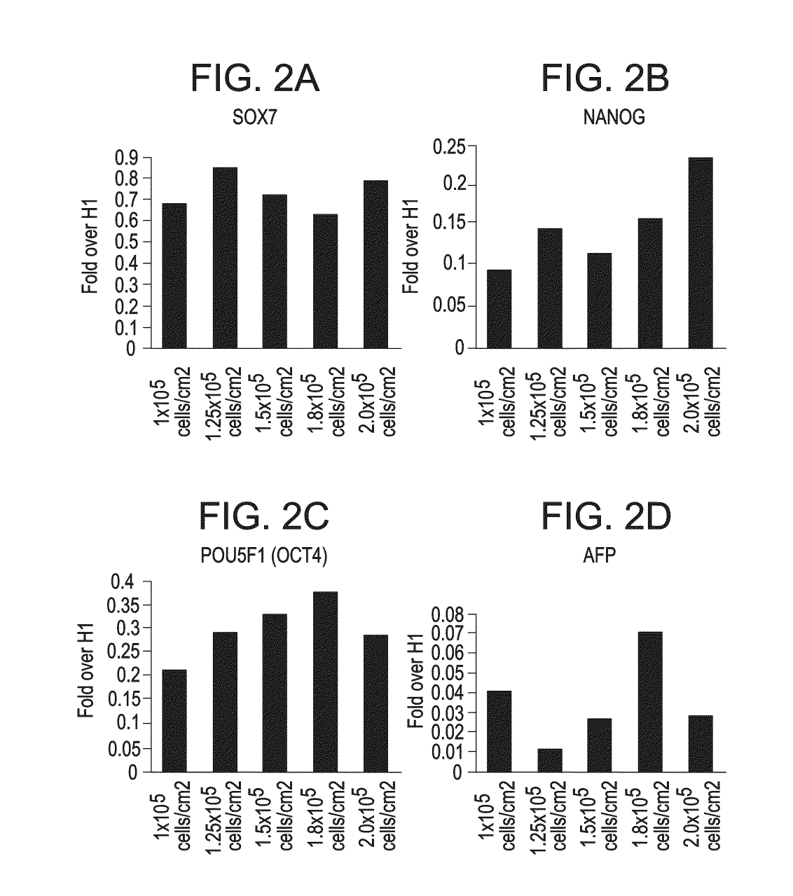

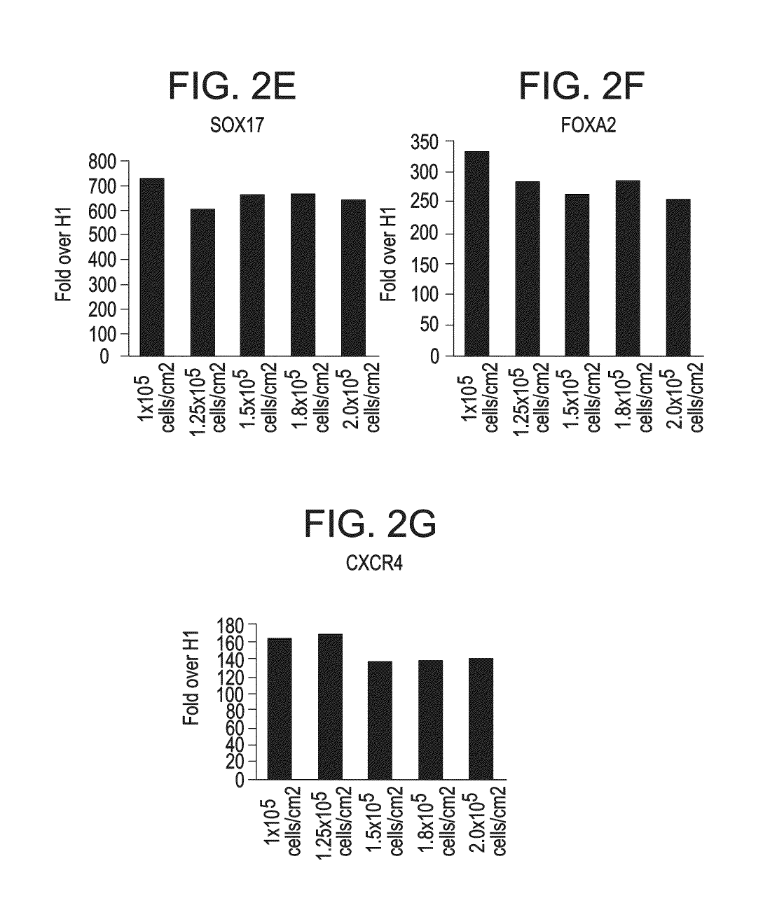

FIG. 2A to FIG. 2G show data from real-time PCR analyses of the expression of the following genes in cells of the human embryonic stem cell line H1 seeded at various densities and subsequently differentiated to DE as outlined in Example 1: SOX7 (FIG. 2A), NANOG (FIG. 2B), OCT4 (FIG. 2C), AFP (FIG. 2D), SOX17 (FIG. 2E), FOXA2 (FIG. 2F), and CXCR4 (FIG. 2G). Consistent with FACS data, there was no significant difference between genes commonly expressed at DE stage (CXCR4, SOX17, FOXA2) for H1 cells seeded at various densities on Matrigel™-coated surfaces. Moreover, initial seeding density did not have a significant impact on genes associated with extra embryonic endoderm (AFP, SOX7) and pluripotentcy related genes (OCT4, Nanog).

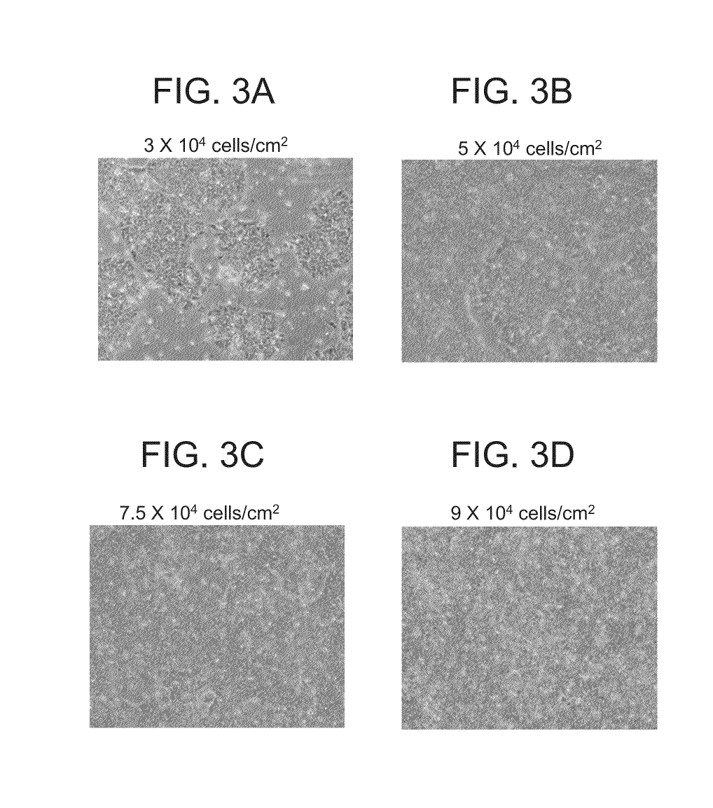

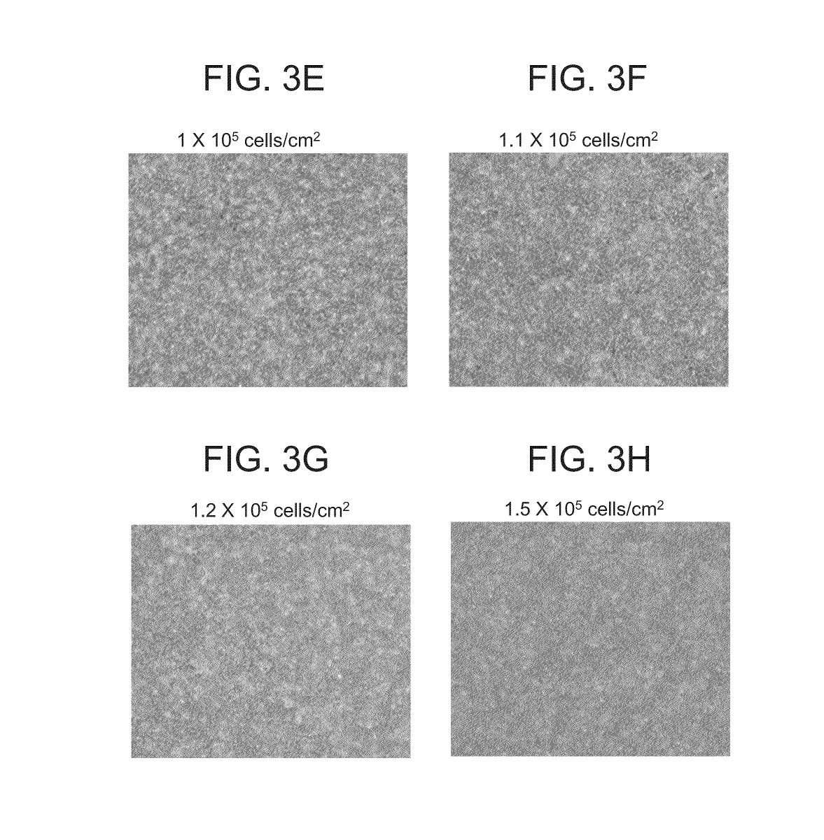

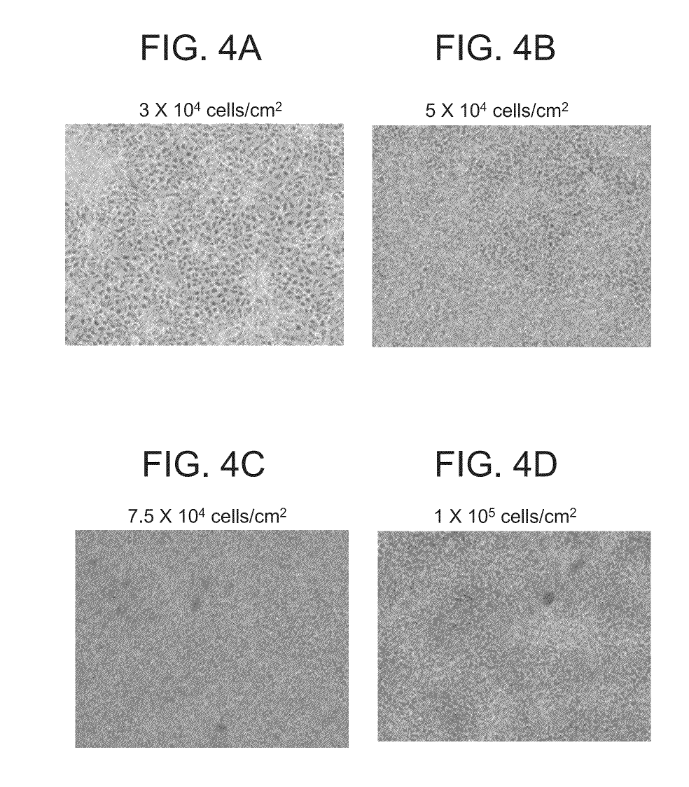

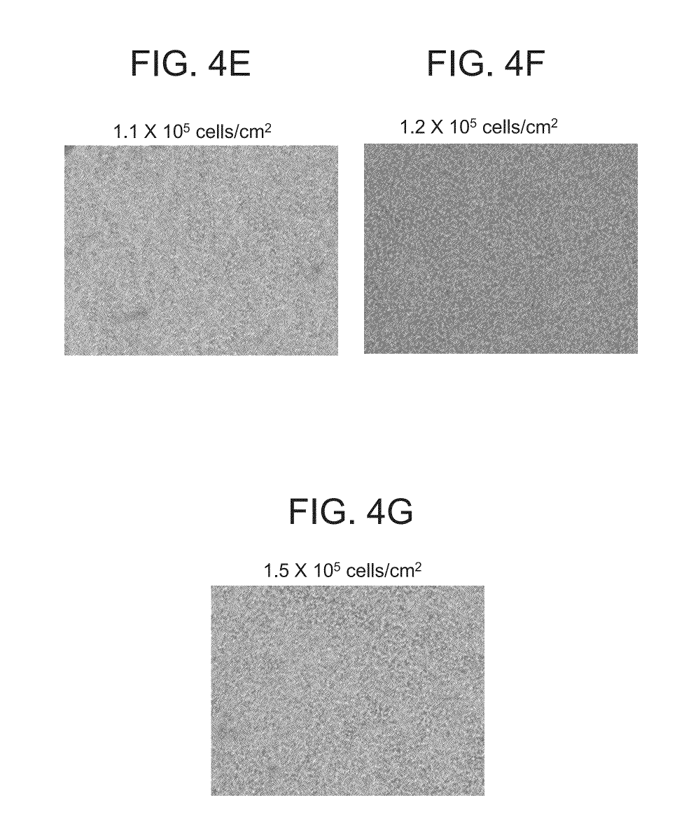

FIGS. 3 and 4 depict phase contrast images of cultures prior to induction of DE (FIG. 3A to FIG. 3G) and 4 days after initiation of differentiation to DE (FIG. 4A to FIG. 4G) for H1 cells seeded at various seeding densities: 3×10⁴ cells/cm² (FIG. 3A and FIG. 4A); 5×10⁴ cells/cm² (FIG. 4A and FIG. 4B); 7.5×10⁴ cells/cm² (FIG. 4A and FIG. 4C); 1×10⁵ cells/cm², FIG. 4D; FIG. 4E, 1.1×10⁵ cells/cm²; FIG. 4F, 1.2×10⁵ cells/cm²; FIG. 4G, 1.5×10⁵ cells/cm². FIG. 4 clearly shows that there was a significant morphological difference for cultures seeded at <1×10⁵ cells/cm² as compared to cultures seeded at higher cell densities. However, this difference did not translate into significant difference in genes/protein associated with DE. Data from this example highlight that the initial seeding density did not significantly impact expression of markers associated with DE. Cultures of ES cells seeded at densities in the range of 0.3-2×10⁵ cells/cm² showed similar efficiencies in differentiation to DE.

### Example 2

### Seeding Density of Embryonic Stem Cells Significantly Affect Expression of Pancreatic Endoderm and Pancreatic Endocrine Markers

This example was carried out to understand if the initial seeding density of ES significantly impacts generation of pancreatic endoderm/endocrine cultures.

Cells of the human embryonic stem cell line H1 (hESC H1) were harvested at various passages (passage 40 to passage 52) and were seeded as single cells at the following densities: 0.5×10⁵ cells/cm2, 0.75×10⁵ cells/cm², 1×10⁵ cells/cm², 1.5×10⁵ cells/cm², 1.8×10^{5 cells/cm}², and 2×10⁵ cells/cm² on MATRIGEL™ (1:30 dilution; BD Biosciences, NJ) coated dishes in MEF-CM (conditioned media) supplemented with 10 μM of Y27632. Forty-eight hours post seeding, cultures were washed and incubated in incomplete PBS (phosphate buffered saline without Mg or Ca) for approximately 30 seconds.

Cultures were differentiated into pancreatic endoderm/endocrine lineages as follows:
a) Stage 1 (Definitive Endoderm (DE)-4 days): Cells were cultured for one day in stage 1 media: MCDB-131 medium (Invitrogen Catalog No. 10372-019) supplemented with 2% fatty acid-free BSA (Proliant Catalog No. 68700), 0.0012 g/ml sodium bicarbonate (SigmaAldrich Catalog No. S3187), 1× GlutaMax™ (Invitrogen Catalog No. 35050-079), 2.5 mM D-Glucose (SigmaAldrich Catalog No. G8769), 1:50000×ITS-X (Invitrogen), 100 ng/ml GDF8 (R&D Systems) and 2.5 μM MCX compound. Cells were then cultured for additional three days in MCDB-131 medium supplemented with 2% fatty acid-free BSA, 0.0012 g/ml sodium bicarbonate, 1× GlutaMax™, 2.5 mM D-Glucose, 100 ng/ml GDF8, and 1:50000×ITS-X.
b) Stage 2 (Primitive gut tube-2 days): Cells were treated for two days with MCDB-131 medium supplemented with 1:50000×ITS-X, 0.1% ALBUMAX BSA (Invitrogen); 0.0012 g/ml sodium bicarbonate; 1× GlutaMax™; 2.5 mM D-Glucose; and 50 ng/ml FGF7, then
c) Stage 3 (Foregut-3 days): Cells were treated with MCDB-131 medium supplemented with a 1:200 dilution of ITS-X; 20 mM Glucose; 1× GlutaMax™; 0.0015 g/ml sodium bicarbonate; 0.1% ALBUMAX BSA; 0.25 μM SANT-1; 20 ng/ml of Activin-A; 2 μM RA; 50 ng/ml FGF7; and 200 nM LDN (BMP receptor inhibitor; Catalog No. 04-0019; Stemgent, CA) for three days.
d) Stage 4 (Pancreatic foregut precursor-3 days): Cells were treated with MCDB-131 medium supplemented with a 1:200 dilution of ITS-X; 20 mM Glucose; 1× GlutaMax™; 0.0015 g/ml sodium bicarbonate; 0.1% ALBUMAX BSA; 0.25 μM SANT-1; 50 nM TPB (PKC activator; Catalog No. 565740; EMD Chemicals, Gibstown, N.J.); 200 nM LDN-193189; 2 μM ALk5 inhibitor (SD-208, disclosed in Molecular Pharmacology 2007, 72:152-161); and 100 nM CYP26A inhibitor (N-{4-[2-Ethyl-1-(1H-1,2,4-triazol-1-yl)butyl]phenyl}-1,3-benzothiazol-2-amine, Janssen, Belgium) for three days.
e) Stage 5 (Pancreatic endoderm/endocrine −3 days): Stage 4 cells were treated with MCDB-131 medium supplemented with a 1:200 dilution of ITS-X; 20 mM Glucose; 1× GlutaMax™; 0.0015 g/ml sodium bicarbonate; 0.1% ALBUMAX BSA; 200 nM LDN-193189; 100 nM CYP26A inhibitor, and 2 μM ALk5 for three days.

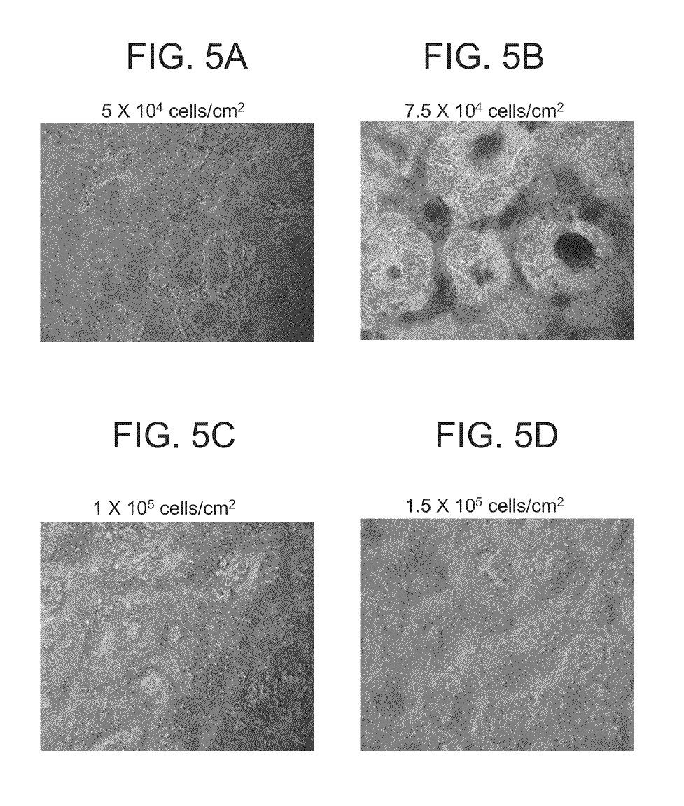

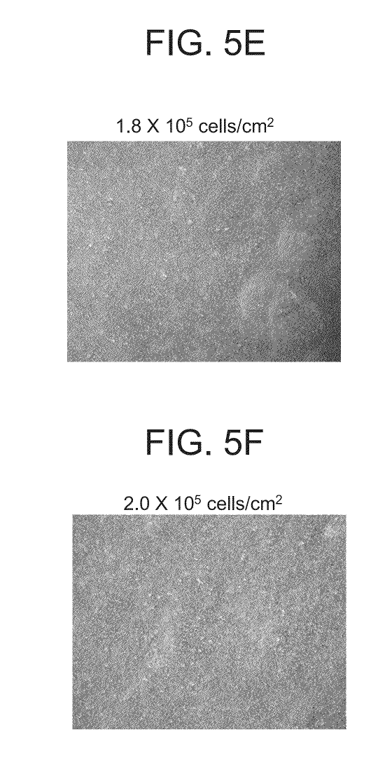

At end of stage 5, phase contrast images were collected for all tested cell densities along with mRNA for PCR analysis of relevant pancreatic endoderm genes. FIG. 5A-5F show phase contrast images of stage 5 cultures that were initially seeded at various cell densities of ES cells: 5×10⁴ cells/cm² (FIG. 5A), 7.5×10⁴ cells/cm² (FIG. 5B), 1×10⁵ cells/cm² (FIG. 5C), 1.5×10⁵ cells/cm² (FIG. 5D), 1.8×10⁵ cells/cm² (FIG. 5E) and 2.0×10⁵ cells/cm² (FIG. 5F). Dramatic heterogeneity of cultures differentiated from cultures seeded at densities less than 1×10⁵ cells/cm² indicates that initial cell density of ES cells significantly impacts morphology of later stage cultures. In particular, cells differentiated from cultures initially seeded at a density higher than 1.5×10⁵ cells/cm² showed a uniform morphology throughout the area of the culture dish.

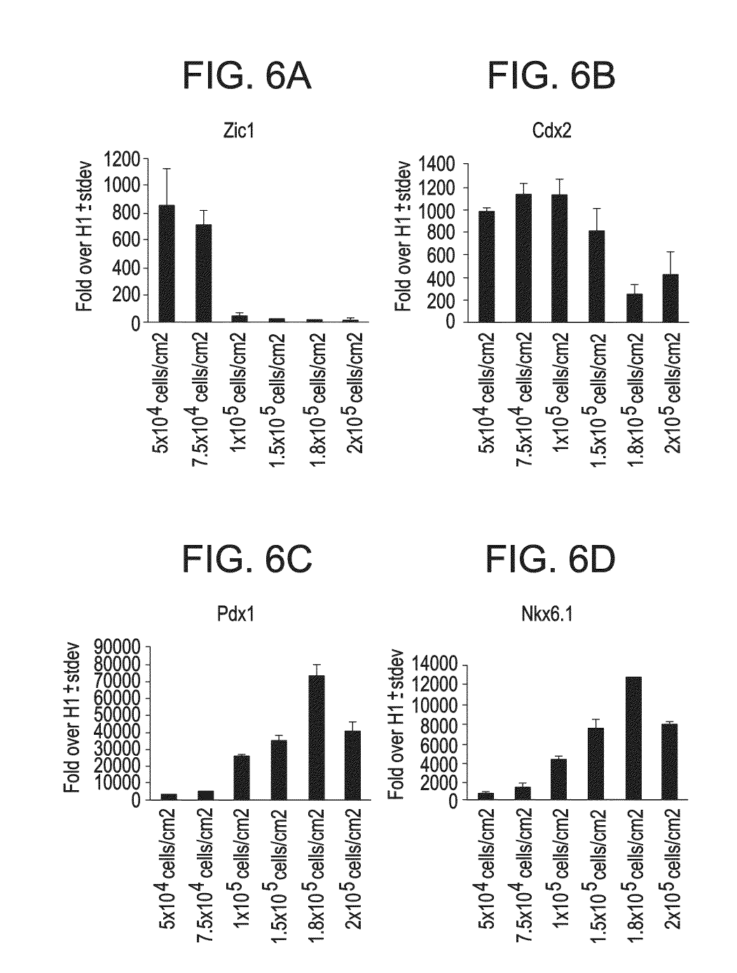

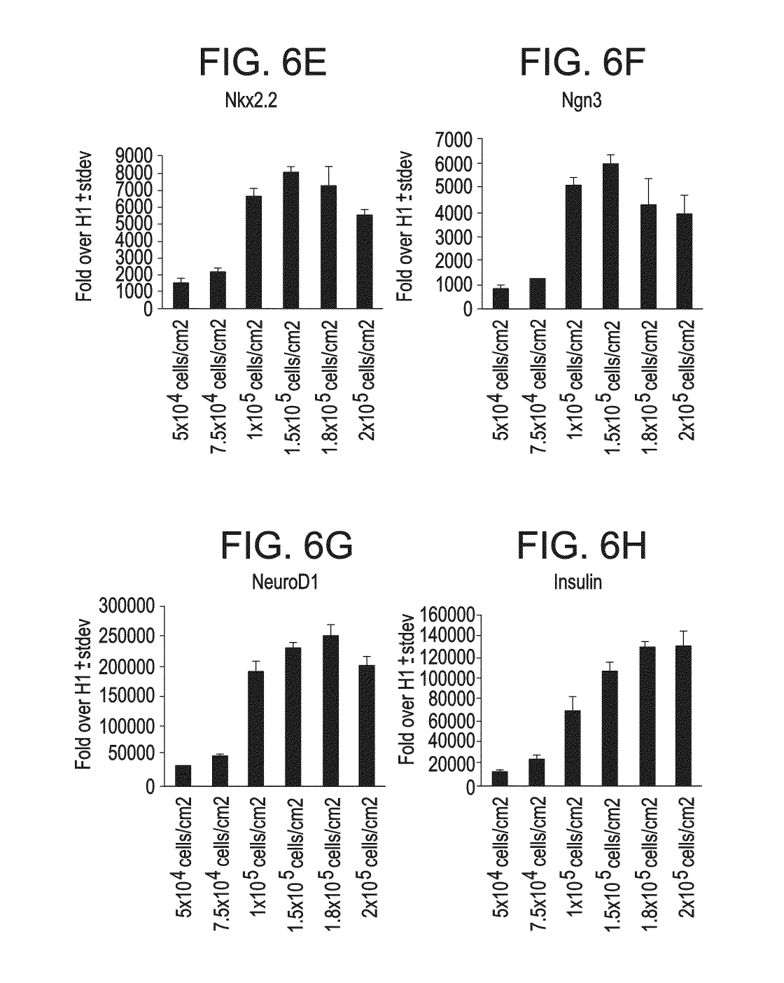

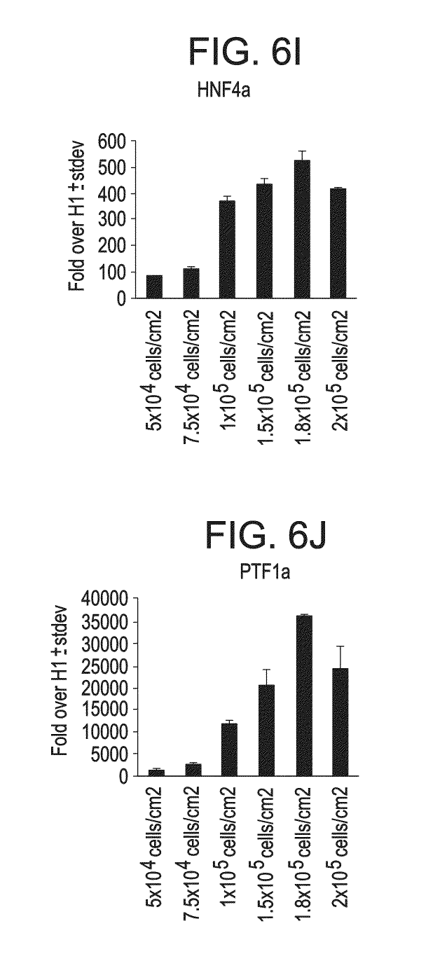

FIG. 6A to FIG. 6J depict data from real-time PCR analyses of the expression of the following genes in cells of the human embryonic stem cell line H1 seeded at various densities and subsequently differentiated to stage 5 as outlined in Example 2: ZIC1 (FIG. 6A), CDX2 (FIG. 6B), PDX-1 (FIG. 6C), NKX6.1 (FIG. 6D), NKX2.2 (FIG. 6E), NGN3 (FIG. 6F), NEUROD (FIG. 6G), insulin (FIG. 6H) HNF4a (FIG. 6I), and PTF1a (FIG. 6J). Unlike the effects observed in Example 1, initial seeding density dramatically affected expression of pancreatic endoderm/endocrine markers. In particular, cells differentiated from cultures with an initial seeding density of less than 1-1.5×10⁵ cells/cm² showed a significant drop in expression of PDX-1, NKX6.1, NGN3, NKX2.2, NeuroD, and insulin while showing upregulation of ectoderm marker ZIC1 and posterior gut marker, CDX2. This data along with data from Example 1 clearly highlight that a high expression of CXCR4 and other DE related genes are not predictive of production of pancreatic endoderm/endocrine genes. Initial seeding density appears to be an important variable in controlling the efficiency of pancreatic endoderm/endocrine cells.

What is claimed is:

1. A method of culturing pluripotent stem cells comprising seeding the pluripotent stem cells on a surface at a seeding density of from about 0.8×10⁵ cells/cm² to about 3.0×10⁵ cells/cm².

2. The method of claim 1, wherein the pluripotent stem cells are embryonic stem cells.

3. The method of claim 2, wherein the embryonic stem cells are human embryonic stem cells.

4. The method of claim 1, wherein the pluripotent stem cells are seeded on a surface comprising Matrigel™.

5. A method of differentiating pluripotent stem cells comprising seeding the pluripotent stem cells on a surface at a density of from about 0.8×10⁵ cells/cm² to about 3.0×10⁵ cells/cm²; and differentiating the pluripotent stem cells to cells expressing markers indicative of definitive endoderm.

6. The method of claim 5, wherein the pluripotent stem cells are embryonic stem cells.

7. The method of claim 6, wherein the embryonic stem cells are human embryonic stem cells.

8. The method of claim 5, wherein the surface where the pluripotent stem cells are seeded comprises Matrigel™.

9. The method of claim 5, wherein the cells expressing markers indicative of definitive endoderm are human.

10. A method of obtaining cells expressing markers indicative of definitive endoderm comprising differentiating pluripotent stem cells into cells expressing markers indicative of definitive endoderm, wherein the pluripotent stem cells have been seeded on a surface at a density of from about 0.8×10⁵ cells/cm² to about 3.0×10⁵ cells/cm².

11. The method of claim 10, wherein the pluripotent stem cells are embryonic stem cells.

12. The method of claim 11, wherein the embryonic stem cells are human embryonic stem cells.

13. The method of claim 10, wherein the surface where the pluripotent stem cells are seeded comprises Matrigel™.

14. The method of claim 10, wherein the cells expressing markers indicative of definitive endoderm are human.

15. A method of differentiating cells expressing markers indicative of definitive endoderm comprising seeding pluripotent stem cells on a first surface at a seeding density sufficient to maximize differentiation efficiency of the pluripotent stem cells; differentiating the pluripotent stem cells into cells expressing markers indicative of definitive endoderm; seeding the cells expressing markers indicative of definitive endoderm at a seeding density sufficient to maximize differentiation efficiency of the cells expressing markers indicative of definitive endoderm; and differentiating the cells expressing markers indicative of definitive endoderm into cells expressing markers indicative of pancreatic endoderm.

16. The method of claim 15, wherein the pluripotent stem cells are seeded on the first surface at a seeding density of from about 0.8×10⁵ cells/cm² to about 3.0×10⁵ cells/cm².

17. The method of claim 15, wherein the cells expressing markers indicative of definitive endoderm are seeded on the second surface at a seeding density of from about 1.5×10⁵ cells/cm² to about 5.0×10⁵ cells/cm².

18. The method of claim 15, wherein the pluripotent stem cells are embryonic stem cells.

19. The method of claim 18, wherein the embryonic stem cells are human embryonic stem cells.

20. The method of claim 15, wherein the first surface comprises Matrigel™.

21. The method of claim 15, wherein the second surface comprises Matrigel™.

22. The method of claim 15, wherein the first surface and the second surface are the same surface.

23. The method of claim 15, wherein the cells expressing markers indicative of definitive endoderm are human.

24. The method of claim 15, wherein the cells expressing markers indicative of pancreatic endoderm are human.

25. A method of differentiating cells expressing markers indicative of definitive endoderm comprising differentiating pluripotent stem cells into cells expressing markers indicative of definitive endoderm; and differentiating the cells expressing markers indicative of definitive endoderm into cells expressing markers indicative of pancreatic endoderm; wherein the pluripotent stem cells have been seeded on a surface at a seeding density of from about 0.8×10⁵ cells/cm² to about 3.0×10⁵ cells/cm².

26. The method of claim 25, wherein the pluripotent stem cells are embryonic stem cells.

27. The method of claim 26, wherein the embryonic stem cells are human embryonic stem cells.

28. The method of claim 25, wherein the surface comprises Matrigel™.

29. The method of claim 25, wherein the cells expressing markers indicative of definitive endoderm are human.

30. The method of claim 25, wherein the cells expressing markers indicative of pancreatic endoderm are human.

31. A method of obtaining cells expressing markers indicative of pancreatic endoderm comprising:
a) seeding pluripotent stem cells on a surface;
b) differentiating the pluripotent stem cells into cells expressing markers indicative of definitive endoderm; and
c) differentiating the cells expressing markers indicative of definitive endoderm into cells expressing markers indicative of pancreatic endoderm.

32. The method of claim 31, wherein the pluripotent stem cells are seeded on a surface at a seeding density of from about 0.8×10⁵ cells/cm² to about 3.0×10⁵ cells/cm².

33. The method of claim 31, further comprising the step of seeding the cells expressing markers indicative of definitive endoderm at a seeding density of from about 1.5×10⁵ cells/cm² to about 5.0×10⁵ cells/cm².

34. The method of claim 31, wherein the pluripotent stem cells are embryonic stem cells.

35. The method of claim 34, wherein the embryonic stem cells are human embryonic stem cells.

36. The method of claim 31, wherein the surface comprises Matrigel™.

37. The method of claim 31, wherein the cells expressing markers indicative of definitive endoderm are human.

38. The method of claim 31, wherein the cells expressing markers indicative of pancreatic endoderm are human.

39. A method of obtaining cells expressing markers indicative of pancreatic endocrine comprising:
a) seeding pluripotent stem cells on a surface;
b) differentiating the pluripotent stem cells into cells expressing markers indicative of definitive endoderm; and
c) differentiating the cells expressing markers indicative of definitive endoderm into cells expressing markers indicative of pancreatic endocrine.

40. The method of claim 39, wherein the pluripotent stem cells are seeded at a seeding density of from about 0.8×10⁵ cells/cm² to about 3.0×10⁵ cells/cm².

41. The method of claim 39, further comprising the step of seeding the cells expressing markers indicative of definitive endoderm at a seeding density of from about 1.5×10⁵ cells/cm² to about 5.0×10⁵ cells/cm².

42. The method of claim 39, wherein the pluripotent stem cells are embryonic stem cells.

43. The method of claim 40, wherein the embryonic stem cells are human embryonic stem cells.

44. The method of claim 39, wherein the surface comprises Matrigel™.

45. The method of claim 39, wherein the cells expressing markers indicative of definitive endoderm are human.

46. The method of claim 39, wherein the cells expressing markers indicative of pancreatic endoderm are human.

47. A method of differentiating cells expressing markers indicative of definitive endoderm comprising seeding cells expressing markers indicative of definitive endoderm on a surface at a seeding density of from about 1.5×10⁵ cells/cm² to about 5.0×10⁵ cells/cm²; and differentiating the cells expressing markers indicative of definitive endoderm into cells expressing markers indicative of pancreatic endoderm.

48. The method of claim 47, wherein the cells expressing markers indicative of definitive endoderm are human.

49. The method of claim 47, wherein the cells expressing markers indicative of pancreatic endoderm are human.

50. A method of differentiating cells expressing markers indicative of definitive endoderm into cells expressing markers indicative of pancreatic endocrine comprising seeding cells expressing markers indicative of definitive endoderm on a surface at a seeding density of from about 1.5×10⁵ cells/cm² to about 5.0×10⁵ cells/cm²; and differentiating the cells expressing markers indicative of definitive endoderm into cells expressing markers indicative of pancreatic endocrine.

51. The method of claim 50, wherein the cells expressing markers indicative of definitive endoderm are human.

52. The method of claim 50, wherein the cells expressing markers indicative of pancreatic endoderm are human.

53. A population of cells differentiated in vitro from human embryonic stem cells, showing a drop in expression of at least one marker selected from PDX-1, NKX6.1, NGN3, NKX2.2, NeuroD, and insulin, and upregulation of ZIC1 and CDX2 when compared to human embryonic stem cells; and wherein the human embryonic stem cells are seeded on a surface at a seeding density of less than 5×10⁵ cells/cm².
